(19) United States
(12) Patent Application Publication
(10) Pub. No.: US 2008/0085536 A1
(43) Pub. Date: Apr. 10, 2008

(54) Production of Cellulose in Halophilic Photosynthetic Prokaryotes (Cyanobacteria)

(75) Inventors: David R. Nobles JR., Austin, TX (US); R. Malcolm Brown JR., Austin, TX (US)

Correspondence Address: CHALKER FLORES, LLP, 2711 LBJ FRWY, Suite 1036, DALLAS, TX, 75234, US

(73) Assignee: Board of Regents, The University of Texas System, Austin, TX (US)

(21) Appl. No.: 11/866,863

(22) Filed: Oct. 3, 2007

Related U.S. Application Data
(60) Provisional application No. 60/849,363, filed on Oct. 4, 2006.

(51) Int. Cl. C12P 21/00; C12N 1/20; C12P 7/20; C12P 7/64; C12P 19/04

(52) U.S. Cl. 435/69.1; 435/101; 435/134; 435/159; 435/252.1; 435/41

(57) ABSTRACT

The present invention includes compositions and methods for making and using halophilic cyanobacterium comprising a spontaneous mutation causing constitutive cellulose biosynthesis, whereby the cyanobacterium is capable of producing cellulose in brine. The compositions and methods of the present invention may be used as a new global crop for the manufacture of cellulose, CO₂ fixation, for the production of alternative sources of conventional cellulose as well as a biofuel and precursors thereof.

### CROSS-REFERENCE TO RELATED APPLICATIONS

This application claims priority to U.S. Provisional Patent Application Ser. No. 60/849,363, filed Oct. 4, 2006, the entire contents of which are incorporated herein by reference.

### STATEMENT OF FEDERALLY FUNDED RESEARCH

This invention was made with U.S. Government support under Contract No. DE-FG02-03R15396 awarded by the Department of Energy. The government has certain rights in this invention.

### TECHNICAL FIELD OF THE INVENTION

The present invention relates in general to the field of simultaneous biosynthesis of non-crystalline cellulose and cellulose II in the sheath of a spontaneous cyanobacterial mutant.

### BACKGROUND OF THE INVENTION

Without limiting the scope of the invention, its background is described in connection with cellulose production.

Cellulose biosynthesis has a significant impact on the environment and human economy. The photosynthetic conversion of CO₂ to biomass is primarily accomplished through the creation of the cellulosic cell walls of plants and algae (Lynd et al., 2002). With approximately 10¹¹ tons of cellulose created and destroyed annually (Hess et al., 1928), this process ameliorates the adverse effects of increased production of greenhouse gasses by acting as a sink for CO₂ (Brown, 2004). Although cellulose is synthesized by bacteria, protists, and many algae; the vast majority of commercial cellulose is harvested from plants.

Timber and cotton are the primary sources of raw cellulose for a number of diverse applications including textiles, paper, construction materials, and cardboard, as well as cellulose derived products such as rayon, cellophane, coatings, laminates, and optical films. Wood pulp from timber is the most important source of cellulose for paper and cardboard. However, extensive processing is necessary to separate cellulose from other cell wall constituents (Klemm et al. 2005; Brown, 2004). Both the chemicals utilized to extract cellulose from associated lignin and hemicelluloses from wood pulp and the waste products generated by this process pose serious environmental risks and disposal problems (Bajpai, 2004). Additionally, the cultivation of other cellulose sources, such as cotton, entails the extensive use of large tracts of arable land, fertilizers and pesticides (both of which require petroleum for their manufacture), and dwindling fresh water supplies for irrigation.

### SUMMARY OF THE INVENTION

The present invention relates in general to cellulose biosynthesis by a marine halophilic cyanobacterium that simultaneously synthesizes non-crystalline cellulose and cellulose II constitutively. The cellulose and derivatives of the cellulose may be used in a wide variety of applications, e.g., large scale cellulose production for production of biofuels. More particularly, the present invention includes constitutive production of an extracellular, cellulose-containing sheath by photosynthetic cyanobacteria capable of growing in brine, facultative heterotrophs, chemoautotrophic, and combinations thereof. The cyanobacteria may also be nitrogen-fixing.

The present invention includes a halophilic cyanobacterium producing cellulose in brine. In one aspect, the cyanobacteria may be a photosynthetic cyanobacterium capable of growing in brine, and wherein the isolated cyanobacteria produce cellulose as part of its extracellular sheath. In another aspect, the extracellular sheath can be digested with cellulose-degrading enzymes. In another aspect, the cellulose and its extracellular sheath can be processed into cellulosic ethanol. In certain examples, the cyanobacterium can produce cellulose at salt concentrations of greater than 3.5% (w/v), or at salt concentrations greater than 6% (w/v). In one aspect, the cyanobacterium is a sub-strain of Agmenellum quadruplicatum UTEX B2268, distinct from cultures of this species Synechococcus sp. PCC 7002 and Synechococcus sp. ATCC 27264. In one aspect, the cellulose and its extracellular sheath is processed as a renewable feedstock for biofuel production, or is CO₂ that is fixed into saccharides and/or carbohydrates while producing cellulose and reduces atmospheric CO₂. In another aspect, the cyanobacterium can produce cellulose without the use of fresh water.

In another embodiment, the present invention includes cyanobacterium, e.g., Agmenellum quadruplicatum, capable of producing cellulose in saline environments. In one aspect, the cyanobacterium is Agmenellum quadruplicatum UTEX B2268. In another aspect, the cyanobacterium produces an extracellular sheath digestible by cellulose-degrading enzymes. In one aspect, the cyanobacterium grows at salt concentrations of greater than 4%.

Another embodiment of the present invention includes a method of producing cellulose with cellulose as part of its extracellular sheath, by placing a halophilic cyanobacterium comprising a portion of an exogenous bacterial cellulose operon sufficient to express bacterial cellulose in brine; growing the halophilic cyanobacterium under conditions that promote cellulose production; and separating the cellulose from the brine. In one aspect, the remaining biomass may be used for food, specialty products, and/or fuel. In one aspect, the separated cellulose and its extracellular sheath are digested with cellulose-degrading enzymes. The method may also include the step of processing the cellulose into monomers. The cellulose and its extracellular sheath can be used alone or separately as a renewable feedstock for biofuel production. In one aspect, the cyanobacterium fixes CO₂ and thus atmospheric CO₂.

Another embodiment of the present invention includes a method of generating carbon credits by placing a halophilic cyanobacterium sufficient to express bacterial cellulose in CO₂-containing brine; generating cellulose with the cyanobacterium, wherein CO₂ is fixed into a cellulose biomass; and calculating the amount CO₂ fixed into the biomass to equate to one or more carbon credit units. In one aspect, the carbon credits may be sold to users that are net producers of CO₂ or other carbon emissions that are looking to counterbalance their emissions with a method to fix those carbon emissions, e.g., in a market that trades carbon credits. In one aspect, the at least one other carbon is fixed into a cellulose biomass and the at least one other carbon's equate to carbon credit units is included in the calculation.

The system for the manufacture of bacterial cellulose may further include growing an exogenous cellulose expressing cyanobacterium adapted for growth in a hypersaline environment, such that the cyanobacterium does not grow in fresh water or the salinity of sea water. The growth of the cyanobacteria in a hypersaline environment may be used as way to limit the potential for unplanned growth of the cyanobacteria outside controlled areas. In one example, the cellulose expressing cyanobacteria of the present invention may be grown in brine ponds obtained from subterranean formation, such a gas and oil fields. Examples of cyanobacteria for use with the system include those that are photosynthetic, nitrogen-fixing, capable of growing in brine, facultative heterotrophs, chemoautotrophic, and combinations thereof. As with the previous embodiments of the present invention, the cellulose genes may even obtained from mosses such as Physcomitriella, algae, ferns, vascular plants, tunicates, gymnosperms, angiosperms, cotton, switchgrass and combinations thereof. The skilled artisan will recognize that it is possible to combine portions of the operons of bacterial with algal, fungal and plant cellulose genes to maximize production and/or change the characteristics of the cellulose.

### BRIEF DESCRIPTION OF THE DRAWINGS

For a more complete understanding of the features and advantages of the present invention, reference is now made to the detailed description of the invention along with the accompanying figures and in which:

FIG. 1. Epifluorescence micrographs of Tinopal labeled Agmenellum quadruplicatum UTEX B2268. (A) Phase contrast—note the filamentous morphotype. (B) Phase contrast combined with fluorescence. (C) Epifluorescence. Note the presence of fluorescent extracellular material in Figures (B) and (C). The fluorescence is most intense at cell junctions.

FIG. 2. CBHI-gold labeling of UTEX B2268 colonies from plates. Micrographs A-D represent progressively higher magnifications of the filamentous morphotype of B2268. Note the CBHI-gold labeling of extracellular sheath, which appears to be primarily composed of non-crystalline cellulose with small aggregates of cellulose II embedded.

FIG. 3. CBHI-gold labeling of Acetic/Nitric insoluble material from B2268. After this treatment only crystalline material remains. (A) CBHI-gold labeling of an acid insoluble extracellular polysaccharide associated with cell envelope. (B) Higher resolution micrograph of the region shown in (A) demonstrating short rodlets characteristic of the cellulose II allomorph remaining after acid treatment. Again, note CBHI-gold labeling. CBHI-gold has affinity for crystalline and non-crystalline cellulose.

FIG. 4 shows a diagram of a production plant that may be used to produce, isolate and process the saccharides produced using the present invention.

FIG. 5 shows photobioreactor design for in situ harvest of cyanobacterial saccharides.

FIG. 6 is a side view of a photobioreactor complex design for in situ harvest of cyanobacterial saccharides.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the terms “continuous method” or “continuous feed method” refer to a fermentation method that includes continuous nutrient feed, substrate feed, cell production in the bioreactor, cell removal (or purge) from the bioreactor, and product removal. Such continuous feeds, removals or cell production may occur in the same or in different streams. A continuous process results in the achievement of a steady state within the bioreactor. As used herein, the term “steady state” refers to a system and process in which all of these measurable variables (i.e., feed rates, substrate and nutrient concentrations maintained in the bioreactor, cell concentration in the bioreactor and cell removal from the bioreactor, product removal from the bioreactor, as well as conditional variables such as temperatures and pressures) are relatively constant over time.

As used herein, the terms “photobioreactor,” “photoreactor,” or “cyanobioreactor,” include a fermentation device in the form of ponds, trenches, pools, grids, dishes or other vessels whether natural or man-made suitable for inoculating the cyanobacteria of the present invention and providing to one or more of the following: sunlight, artificial light, salt, water, CO₂, H₂O, growth media, stirring and/or pumps, gravity or force fed movement of the growth media. The product of the photobioreactor will be referred to herein as the “photobiomass”. The “photobiomass” includes the cyanobacteria, secreted materials and mass formed into, e.g., cellulose whether intra or extracellular.

As used herein, the terms “bioreactor,” “reactor,” or “fermentation bioreactor,” include a fermentation device that includes of one or more vessels and/or towers or piping arrangement, which includes the Continuous Stirred Tank Reactor (CSTR), Immobilized Cell Reactor (ICR), Trickle Bed Reactor (TBR), Bubble Column, Gas lift Fermenter, Static Mixer, or other device suitable for gas-liquid contact. A fermentation bioreactor for use with the present invention includes a growth reactor which feeds the fermentation broth to a second fermentation bioreactor, in which most products, e.g., alkanols or furans are produced. In some cases, the gaseous byproduct of fermentation, e.g., CO₂, can be pumped back into the photobioreactor to recycle the gas and promote the formation of saccharides by photosynthesis. To the extent that heat is generated during the process of recovering the products of the fermentation, etc., the heat can also be used to promote cyanobacterial cell growth and production of saccharides.

As used herein, the term “nutrient medium” refers to conventional cyanobacterial growth media that includes sufficient vitamins, minerals and carbon sources to permit growth and/or photosynthesis of the cellulose producing cyanobacteria of the present invention. Components of a variety of nutrient media suitable to the use of this invention are known and reported in e.g., Cyanobacteria, Volume 167: (Methods in Enzymology) (Hardcover), by John N. Abelson Melvin I. Simon and Alexander N. Glazer (Editors), Academic Press, New York (1988).

As used herein, the term “cell concentration” refers to the dry weight of cyanobacteria per liter of sample. Cell concentration is measured directly or by calibration to a correlation with optical density.

As used herein, the term “saccharide production” refers to the amount of mono-, di-, oligo or polysaccharides produced by the modified-cyanobacteria of the present invention that produce saccharides by fixing carbon such as CO₂ by photosynthesis into the saccharides. One distinct advantage of the present invention is that the cyanobacteria do not produce lignin along with the production of the cellulose and other saccharides that can be used a feed-stock for fermentation and other bioreactors that convert the saccharides into, e.g., ethanol or other synfuels.

In operation, the present invention may use any of a variety of known fermentation process steps, compositions and methods for converting the saccharides into useful products, e.g., lignin-free cellulose, alkanols, furans and the like. One non-limiting example of a process for producing ethanol by fermentation is a process that permits the simultaneous saccharification and fermentation step by placing the saccharide source at a temperature of above 34° C. in the presence of a glucoamylase and a thermo-tolerant yeast.

In this example, the following main process stages may be included saccharification (if necessary), fermentation and distillation. One particular advantage of the present invention is that it eliminates a variety of processing steps, including, milling, bulk-fiber separations, recovery or treatments for the control or elimination of lignin, water removal, distillation and burning of unwanted byproducts. Any of the process steps of alcohol production may be performed batchwise, as part of a continuous flow process or combinations thereof.

Saccharification. To produce mono- and di-saccharides from the lignin-free cellulose of the present invention the cellulose can be metabolized by cellulases that provide the yeast with simple saccharides. This “saccharification” step include the chemical or enzymatic hydrolysis of long-chain oligo and polysaccharides by enzymes such as cellulase, glucoamylases, alpha-glucosidase, alkaline, acid and/or thermophilic alpha-amylases and if necessary phytases.

Depending on the length of the polysaccharides, enzymatic activity, amount of enzyme and the conditions for saccharification, this step may last up to 72 hours. Depending on the feedstock, the skilled artisan will recognize that saccharification and fermentation may be combined in a simultaneous saccharification and fermentation step.

Fermentation. Any of a wide-variety of known microorganism may be used for the fermentation, fungal or bacterial. For example, yeast may be added to the feedstock and the fermentation is ongoing until the desired amount of ethanol is produced; this may, e.g., be for 24-96 hours, such as 35-60 hours. The temperature and pH during fermentation is at a temperature and pH suitable for the microorganism in question, such as, e.g., in the range about 32-38° C., e.g. about 34° C., above 34° C., at least 34.5° C., or even at least 35° C., and at a pH in the range of, e.g., about pH 3-6, or even about pH 4-5. The skilled artisan will recognize that certain buffers may be added to the fermentation reaction to control the pH and that the pH will vary over time.

The use of a feed stock that includes monosaccharides and disaccharides, in addition to the use of thermostable acid alpha-amylases or a thermostable maltogenic acid alpha-amylases and invertases in the saccharification step may make it possible to improve the fermentation step. When using a feedstock that includes large amounts of saccharides such as glucose and sucrose, for the production of ethanol it may be possible to reduce or eliminate the need for the addition of glucoamylases in the fermentation step or prior to the fermentation step.

Distillation. To complete the manufacture of final products from the saccharides made by the cyanobacterial fixation of CO₂ of the present invention, the invention may also include recovering the alcohol (e.g., ethanol). In this step, the alcohol may be separated from the fermented material and purified with a purity of up to e.g. about 96 vol. % ethanol can be obtained by the process of the invention.

Several specific enzymes and methods may be used to improve the recovery of energy containing molecules from the present invention. The enzymes improve the saccharification and fermentation steps by selecting their most efficient activity as part of the processing of the products of the saccharide producing modified cyanobacteria of the present invention.

In one example, a thermo tolerant cellulase may be introduced into the reactor to convert cellulose produced by the cyanobacteria of the present invention into monosaccharides, which will mostly be glucose. Examples of thermophilic cellulases are known in the art as taught in, e.g., U.S. Patent Application No 20030104522 filed by Ding, et al. that teach a thermal tolerant cellulase from Acidothermus cellulolyticus. Yet another example is taught by U.S. Patent Application No. 20020102699, filed by Wicher, et al., which teaches variant thermostable cellulases, nucleic acids encoding the variants and methods for producing the variants obtained from Rhodothermus marinus. The relevant portions of each are incorporated herein by reference.

Acid cellulase may be obtained commercially from manufacturers such as Ideal Chemical Supply Company, Memphis Tenn., USA; Americos Industries Inc., Gujarat, India; or Rakuto Kasei House, Yokneam, Israel, Novozyme, Denmark. For example, the acid cellulase may be provided in dry, liquid or high-active abrasive form, as is commonly used in the denim acid washing industry using techniques known to the skilled artisan. For example, Americos Cellscos 450 AP is a highly concentrated acid cellulase enzyme produced using a genetically modified strains of Trichoderma reesii. Typically, the acid cellulases function in a pH range or 4.5-5.5.

Microorganisms used for fermentation. One example of a microorganism for use with the present invention is a thermo-tolerant yeast, e.g., a yeast that when fermenting at 35° C. maintains at least 90% of the ethanol yields and 90% of the ethanol productivity during the first 70 hours of fermentation, as compared to when fermenting at 32° C. under otherwise similar conditions. One example of a thermotolerant yeast is a yeast that is capable of producing at least 15% V/V alcohol from a corn mash comprising 34.5% (w/v) solids at 35° C. One such thermo-tolerant yeast is Red Star®/Lesaffre Ethanol Red (commercially available from Red Star®/Lesaffre, USA, Product No. 42138). The ethanol obtained using any known method for fermenting saccharides (mono, di-, oligo or poly) may be used as, e.g., fuel ethanol, drinking ethanol, potable neutral spirits, industrial ethanol or even fuel additives.

Examples of ethanol fermentation from sugars are well-known in the art as taught by, e.g., U.S. Pat. No. 4,224,410 to Pemberton, et al. for a method for ethanol fermentation in which fermentation of glucose and simultaneous-saccharification fermentation of cellulose using cellulose and a yeast are improved by utilization of the yeast Candida brassicae, ATCC 32196; U.S. Pat. No. 4,310,629 to Muller, et al., that teaches a continuous fermentation process for producing ethanol in which continuous fermentation of sugar to ethanol in a series of fermentation vessels featuring yeast recycle which is independent of the conditions of fermentation occurring in each vessel is taught; U.S. Pat. No. 4,560,659 to Asturias for ethanol production from fermentation of sugar cane that uses a process for fermentation of sucrose wherein sucrose is extracted from sugar cane, and subjected to stoichiometric conversion into ethanol by yeast; and U.S. Pat. No. 4,840,902 to Lawford for a continuous process for ethanol production by bacterial fermentation using pH control in which a continuous process for the production of ethanol by fermentation of an organism of the genus Zymomonas is provided. The method of Lawford is carried out by cultivating the organism under substantially steady state, anaerobic conditions and under conditions in which ethanol production is substantially uncoupled from cell growth by controlling pH in the fermentation medium between a pH of about 3.8 and a pH less than 4.5; and K A Jacques, T P Lyons & D R Kelsall (Eds) (2003), The Alcohol Textbook; 4^{TH} Edition, Nottingham Press; 2003. The relevant portions of each of which are incorporated herein by reference.

One of ordinary skill in the art would recognize that the quantity of yeast to be contacted with the photobiomass will depend on the quantity of the photobiomass, the secreted portions of the photobiomass and the rate of fermentation desired. The yeasts used are typically brewers' yeasts. Examples of yeast capable of fermenting the photobiomass include, but are not limited to, Saccharomyces cerevisiae and Saccharomyces uvarum. Besides yeast, genetically altered bacteria know to those of skill in the art to be useful for fermentation can also be used. The fermenting of the photobiomass is conducted under standard fermenting conditions.

Separating of the ethanol from the fermentation can be achieved by any known method (e.g. distillation). The separation can be performed on either or both the liquid and solid portions of the fermentation solution (e.g., distilling the solid and liquid portions), or the separation can just be performed on the liquid portion of the fermentation solution (e.g., the solid portion is removed prior to distillation). Ethanol isolation can be performed by a batch or continuous process. The separated ethanol, which will generally not be fuel-grade, can be concentrated to fuel grade (e.g., at least 95% ethanol by volume) via additional distillation or other methods known to those of skill in the art (e.g., a second distillation).

The level of ethanol present in the fermentation solution can negatively affect the yeast/bacteria. For example, if 17% by volume or more ethanol is present, then it will likely begin causing the yeast/bacteria to die. As such, ethanol can be separated from the fermentation solution as the ethanol levels (e.g., 12, 13, 14, 15, 16, to 17% by volume (ethanol to water)) that may kill the yeast or bacteria are reached. Ethanol levels can be determined using methods known to those of ordinary skill in the art.

The fermentation reaction can be run multiple times on the photobiomass or portions thereof. For example, once the level of ethanol in the initial fermentation reactor reaches 12-17% by volume, the entire liquid portion of the fermentation solution can be separated from the biomass to isolate the ethanol (e.g., distillation). The “once-fermented” photobiomass can then be contacted with water, additional enzymes and yeast/bacteria for additional fermentations, until the yield of ethanol is undesirably low. Factors that the skilled artisan will use to determine the number of fermentations include: the amount of photobiomass remaining in the vessel; the amount of carbohydrate remaining, the type of yeast or bacteria, the temperature, pH, salt concentration of the media and overall ethanol yield. If any carbohydrates remain, then the remaining photobiomass is removed from the vessel.

Generally, it is desirable to isolate or harvest the yeast/bacteria from the fermentation reaction for recycling. The method of harvesting will depend upon the type of yeast/bacteria. If the yeast/bacteria are top-fermenting, they can be skimmed off the fermentation solution. If the yeast/bacteria are bottom-fermenting, they can be removed from the bottom of the tank.

Often, a by-product of fermentation is carbon dioxide, which is readily recycled into the photobioreactor for fixation into additional saccharides. During the fermentation process, it is expected that about one-half of the decomposed starch will be discharged as carbon dioxide. This carbon dioxide can be collected by methods known to those of skill in the art (e.g., a floating roof type gas holder) and is supplied back into the photobioreactor pool or pools. In colder climates, the heat that may accompany the carbon dioxide will help in the growth of the cyanobacterial pools.

One advantage of the present invention is that it provides a novel CO₂ fixation method for the recycling of environmental greenhouse gases. The present invention provides a source of substrate for cellulose production from carbon dioxide that is fixed into sugar by photosynthesis, thereby removing a major barrier limiting large global scale production of cellulose. If successful on a large scale, this new global cellulose crop will sequester CO₂ from the air, thus reducing the potential greenhouse gas responsible for global warming. Another benefit of the present invention is that forests and cotton crops, the present sources for cellulose, may not be needed in the future, thus freeing the land to allow regeneration of forests and use of cropland for other needs.

Microbial cellulose stands as a promising possible alternative to traditional plant sources. The a proteobacterium Acetobacter xylinum (synonym Gluconacetobacter xylinum [Yamada et al., 1997]) is the most prolific of the cellulose producing microbes. The NQ5 strain (Brown and Lin, 1990) is capable of converting 50% of glucose supplied in the medium into an extracellular cellulosic pellicle (R. Malcolm Brown, Jr., personal communication). Although it possesses the same molecular formula as cellulose derived from plant sources, microbial cellulose has a number of distinctive properties that make it attractive for diverse applications. The cellulose synthesized by A. xylinum is “spun” into the growth medium as highly crystalline ribbons with exceptional purity, free from the contaminating polysaccharides and lignin found in most plant cell walls (Brown et al., 1976). The resulting membrane or pellicle is composed of cellulose with a high degree of polymerization (2000-8000) and crystallinity (60-90%) (Klemm et al., 2005). Contaminating cells are easily removed, and relatively little processing is required to prepare membranes for use. In its never-dried state, the membrane displays exceptional strength and is highly absorbent, holding hundreds of times its weight in water (White and Brown, 1989). A. xylinum cellulose is therefore, well suited as a reinforcing agent for paper and diverse specialty products (Shah and Brown, 2005; Czaja et al., 2006; Tabuchi et al., 2005; Helenius et al., 2006).

In one example, the acsAB genes from the cellulose synthase operon of or the gram negative bacterium, Acetobacter xylinum (=Gluconacetobacter xylinus) under control of a lac promoter have been integrated into the chromosome of a photosynthetic cyanobacterium, Synechococcus leopoliensis. UTCC 100 may be integrated into halophilic cyanobacteria.

Despite it superior quality, the use of microbial cellulose as a primary constituent for large scale use in common applications such as the production of construction materials, paper, or cardboard has not been economically feasible. The root cause for the expense of microbial cellulose production is the heterotrophic nature of A. xylinum. Bacterial cultures must be supplied with glucose, sucrose, fructose, glycerol, or other carbon sources produced by the cultivation of plants. Increased distance from the primary energy source is inherently less efficient and inevitably leads to increased cost of production when compared with phototrophic sources. As such, the present invention provides compositions and methods for the manufacture of a new global crop that may be used for energy production and removal of the greenhouse gas CO₂ using an environmentally acceptable natural process that requires little or no energy input for manufacture.

Unlike A. xylinum, cyanobacteria require no fixed carbon source for growth. Additionally, many cyanobacteria are capable of nitrogen fixation, which would eliminate the need for fertilizers necessary for cellulose crops like cotton. Furthermore, many cyanobacteria are halophilic, that is, they can grow in a range of brackish to hypersaline environments. This feature, in combination with N-fixation, will allow non-arable, sun-drenched areas of the planet to provide the extensive surface areas for the growth and harvest of cellulose made using the compositions and methods of the present invention on a global scale.

Cyanobacterial cellulose can be used in diverse applications where a combination of products is simultaneously made from photosynthesis. Value added products may include pharmaceuticals and/or vaccines, vitamins, industrial chemicals, proteins, pigments, fatty acids and their derivatives (such as polyhydroxybutyrate), acylglycerols (as precursors for biodiesel), as well as other secondary metabolites. The present invention permits large scale production of cellulose, proteins and other products that may be grown and harvested. In fact, wide application of the cells themselves for glucose and cellulose is encompassed by the present invention. The cellulose producing cyanobacteria of the present invention may be utilized for energy recycling and recovery, that is, the cells may be dried and burned to power downstream processes in a manner similar to the use of bagasse in the sugar cane industries.

Culture Conditions. Two strains of Agmenellum quadruplicatum (PR-6), one obtained as Agmenellum quadruplicatum UTEX B2268 from the University of Texas at Austin Culture Collection of Algae and a second obtained from the American Type Culture Collection as Synechococcus sp. ATCC 27264, were maintained at 24° C. with 12 hour light/dark cycles. Cultures were grown using medium A as previously described (Stevens et al, 1973). Cell concentrations were monitored by measurement of the optical density at 750 nm (OD₇₅₀).

Celluclast Digestions. Celluclast (Sigma C2730) was diluted 1:1 in 20 mM Sodium Acetate Buffer, pH 5.2 and sterilized by passage through a 0.2 um filter (Pall Life Sciences PN 4433). 50 ml cultures of UTEX B2268 and ATCC 27264 were grown to stationary phase. 40 ml of each culture was centrifuged (10 min, RT, 1,744×g) in an IEC clinical centrifuge. The supernatants were discarded and the pellets resuspended in 10 mM Sodium Acetate Buffer, pH 5.2. For buffer-only samples, 250 ul aliquots were transferred to 1.5 ml Eppendorf tubes. For Celluclast digestions, 247.5 ul of resuspended cells and 2.5 ul of sterilized Celluclast were combined in 1.5 ul eppendorf tubes. Enzyme blanks containing only Celluclast and buffer were also prepared. The tubes were placed on a rotisserie and incubated overnight at 30° C. under constant illumination.

Glucose Assays. After overnight incubation, cells were pelleted by centrifugation (5 min, RT, 14,000 rpm) in a microcentrifuge. The supernatant was carefully pipetted off the cell pellet and retained for the glucose assay. Glucose concentration was measured using the hexokinase-glucose, 6-phosphate dehydrogenase enzymatic assay (Sigma G3293). Assays were performed with 50 ul of supernatant per reaction following the manufacturer's instructions. The glucose concentration in the Celluclast enzyme blanks was subtracted from the overall glucose concentration in the experimental samples to obtain the final glucose concentrations.

Light Microscopy. Samples of UTEX B2268 were scraped from agar plates and suspended in growth medium supplemented with 100 uM Tinopal for epifluorescence microscopy. Epifluorescence microscopy was performed with an excitation wavelength of 365 nm.

TEM. Acetic/Nitric Treated Samples. UTEX B2268 colonies collected from plates were suspended in 1 ml of Acetic/Nitric reagent (Updegraff, 1969) and placed in an 80° C. water bath for 1 hour. Insoluble material was collected by centrifugation (10 min, RT, 14,000 rpm) in a microcentrifuge. The pellets were washed to with glass distilled H₂O until a neutral pH was obtained.

CBHI-gold labeling and Negative Staining. Acetic/Nitric insoluble material and UTEX B2268 colonies suspended in glass distilled H₂O were labeled with CBHI-gold and negatively stained as previously described (Nobles et al, 2001).

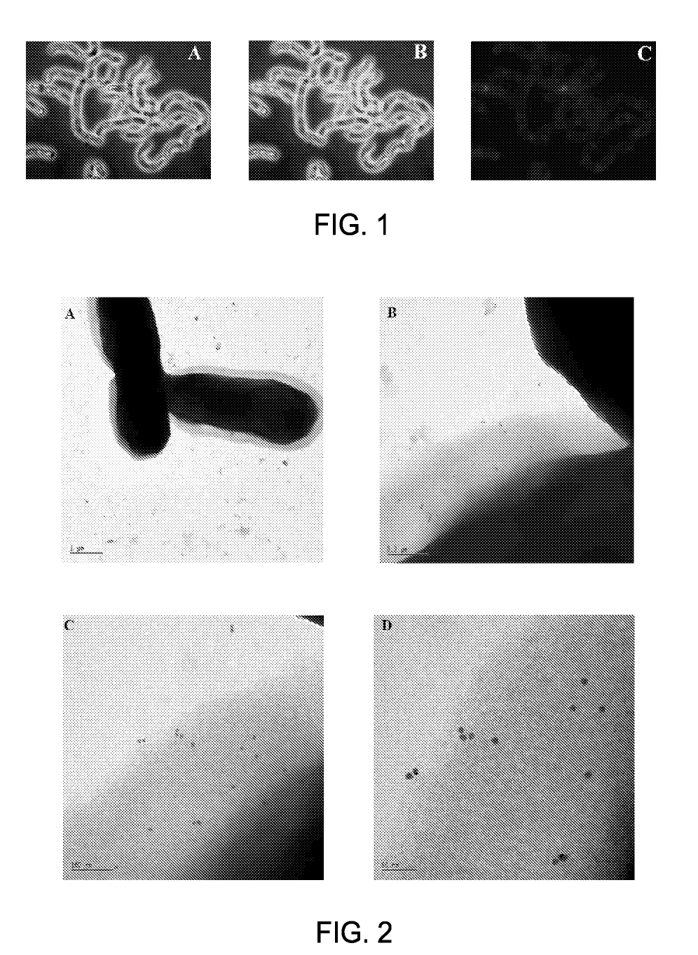

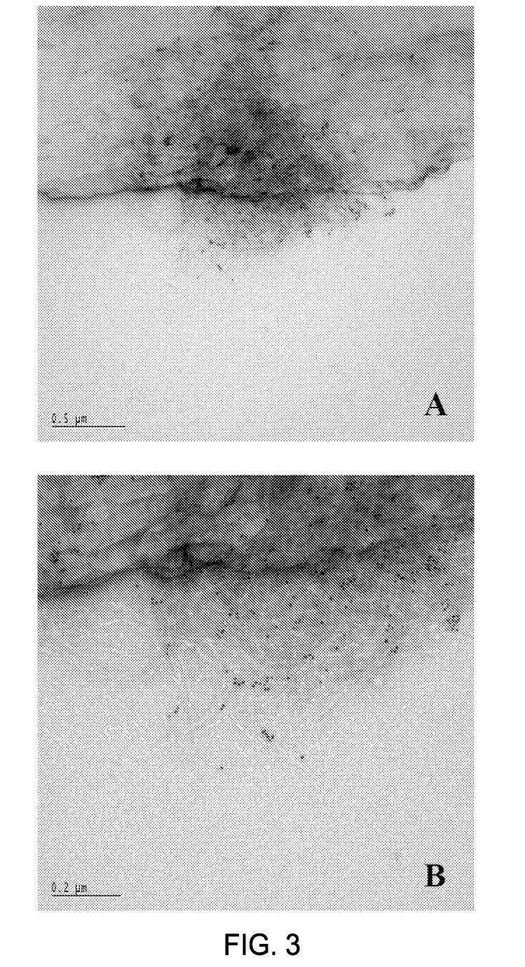

Epifluorescence microscopy was used to demonstrate the presence of Tinopal-labeled, extracellular sheath material associated with the filamentous morphotype of Agmenellum quadruplicatum UTEX B2268 (FIG. 1). No labeling was observed with ATCC 27264 (Results not shown). CBHI-gold labeling of the extracellular material confirms the presence of cellulose as a component of the sheath of B2268 (FIG. 2). The presence of Acetic/Nitric insoluble material labeled with CBHI-gold demonstrates the presence of crystalline cellulose (FIG. 2). Interestingly, the morphology of this cellulose is consistent with the cellulose II allomorph. Cellulose II is rarely observed in nature: its synthesis has only been described in the marine alga Halicystis (Roelosfsen, 1959), the gram positive bacterium Sarcina ventriculi (Roberts, 1991), and by mutants of A. xylinum (Saxena et al, 1994). Definitive identification of cellulose II in the sheath of B2268 will require confirmation by x-ray and/or electron diffraction.

The difference in composition of the extracellular material of ATCC 27264 and UTEX B2268 demonstrated by Tinopal labeling were confirmed by the results of hydrolysis by Celluclast. The data in Table I show that incubation with Celluclast yielded 17.8 mg of glucose liter⁻¹ in B2268, while no glucose liberation was observed in 27264. This is consistent other observed phenotypic differences in these two strains: (1) ATCC 27264 has a higher optimal growth temperature than UTEX B2268 (27264 is reported to prefer 38° C. while B2268 grows optimally at temperatures <30° C.), (2) B2268 constitutively demonstrates the filamentous morphotype—in 27264, this morphotype is only observed at growth below optimal growth temperature, and (3) B2268 maintains a yellowish pigmentation that is associated with nitrogen starvation in 27264. These phenotypic differences can likely be explained by a long separation under different culture and maintenance conditions. The American Type Culture Collection does not maintain its strains in continuous culture in order to prevent genetic drift. However, the University of Texas at Austin Culture Collection of Algae maintains its strains in continuous culture under low light at 20+/−1° C. These conditions may have contributed to selection for one or more mutations allowing genetic drift from the original strain.

Table 1—Glucose liberated from A. quadruplicatum strains post incubation with Celluclast. Values representing cell concentrations, cell mass, and glucose production by A. quadruplicatum UTEX B2268 and ATCC 27264. Optical densities and wet weights were recorded prior to Celluclast digestion. The glucose concentration in mg/ml was measured from aliquots of cell suspensions resulting from the concentration of 40 ml of liquid culture into 1 ml of Celluclast digestion buffer.

TABLE 1
Glucose liberated from A. quadruplicatum strains.

| | | | Glucose | mg Glucose | mg Glucose |
|---|---|---|---|---|---|
| Strain | OD₇₅₀ | Wet weight (g) | (mg/ml) | g̅ w̅e̅t̅ w̅e̅i̅g̅h̅t̅ | liter |
| B2268 | 1.41 | 0.14 | 0.71 | 5.1 | 17.8 |
| 27264 | 1.96 | 0.33 | 0.00 | 0.0 | 0.0 |

Assuming a lossless scale-up, the data in Table 1 project a yield of approximately 200 gallons of ethanol acre foot⁻¹ year⁻¹. This is significantly less than predicted yields for switchgrass (1150 gallons acre⁻¹ year⁻¹). However Agmenellum quadruplicatum possesses several advantageous characteristics which may allow it to be competitive with land-based crops: (1) It possesses a rapid generation time (as short as 4 hours [Sakamoto and Bryant, 1998]), (2) It grows in a wide range of salinities (0.1 to 1.5 M NaCl [Tel-Or et al, 1986]), and (3) the cellulose synthesized by this organism can be hydrolyzed by cellulytic enzymes without the pretreatment procedures required when utilizing lignocellulosic feedstocks, such as switchgrass, for ethanol production. Additionally, this organism is amenable to genetic manipulation by both natural transformation and conjugation. Thus, the potential for increased production by genetic manipulation exists.

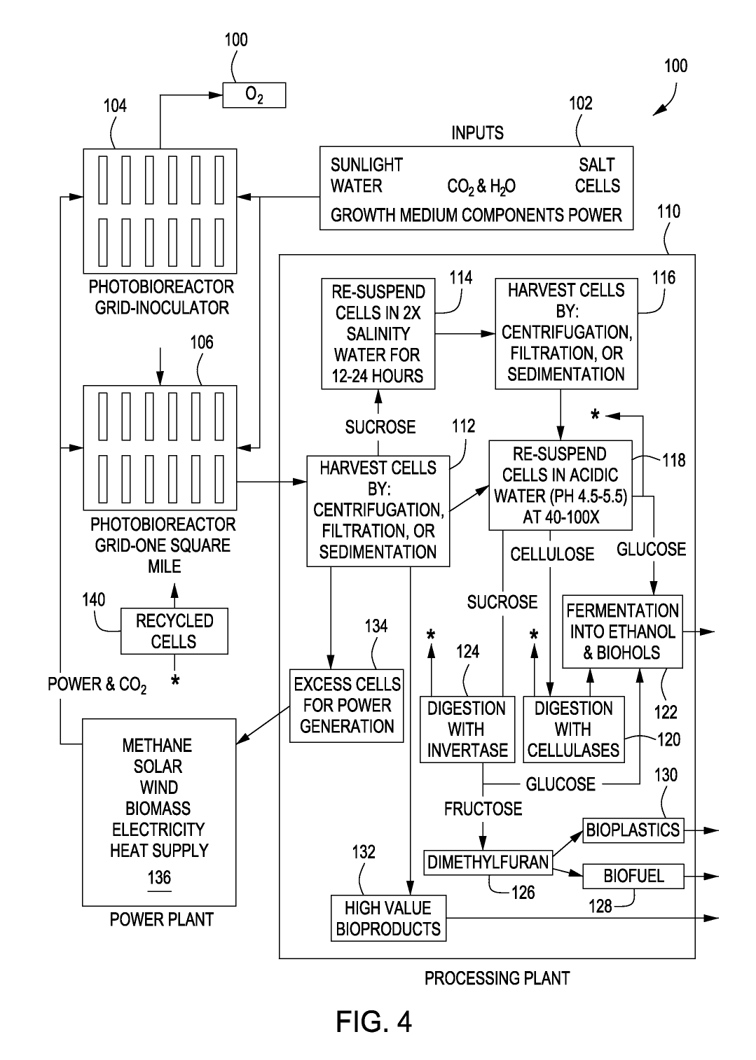

FIG. 4 shows on example of a photobioreactor system 100 of the present invention. First, inputs 102 for the photobioreactor system may include: sunlight, artificial light, salt, water, CO₂ modified-cyanobacterial cells of the present invention, growth medium components and if necessary a source of power to move the components (e.g., pumps or gravity). Next, the inputs 102 and inoculated into a photobioreactor grid 104 that is used to grow the modified-cyanobacteria in size and number, to test for saccharide production and to reach a sufficiently high enough concentration to inoculate the operating photobioreactor 106. The photobioreactor 106 may be a pool or pool(s), trench or other vessel, indoor or outdoor that is used to grow and harvest a sufficient volume of photobiomass for subsequent processing in, e.g., processing plant 110. In one example, the photobioreactor 106 may be a grid of pools of one square mile (or larger) that may be used in parallel or in series to produce the photobiomass. Depending on the geographical location of the photobioreactor 106, the water may be saltwater or brine obtained from a sea that is gravity fed into the pools. Gravity or pumping may be used, however, gravity has the advantage that it does not require additional energy to move the photobiomass from pool to pool and even into the processing plant. In fact, in certain embodiments the entire system may be gravity fed with the final products gravity fed into underground rivers that return to the sea or ocean.

The processing plant 110 includes a cell harvested 112, which may allows the isolation of the photobiomass by, e.g., centrifugation, filtration, sedimentation, creaming or any other method for separating the photobiomass, the modified-cyanobacterial cells and the liquid. For the isolation of sucrose, the cells may be resuspended in medium with an increased salinity 114 (e.g., 2× the salinity) followed by a second harvesting step 116. The twice-harvested cells are then resuspended under acidic conditions (e.g., pH 4.5-5.5) at 40 to 100× the concentration and the sucrose is secreted by the modified-cyanobacteria. If glucose is preferred, the once harvested cells are resuspended under acidic conditions 118 and glucose is secreted. In addition, whether sucrose or glucose is secreted, cellulose is also harvested from the modified-cyanobacteria, which may be further digested by cellulases 120. Glucose and digested cellulose can then be fermented into ethanol or other alkanols (alkyl alcohols).

If sucrose is secreted and obtained, then the sucrose can be converted into dimethylfuran and glucose by invertase 124. The methylfuran 12 can then be used for bioplastic 130 or biofuel 128 production. Glucose that is obtained after the invertase reaction 124 can be directed back into the fermentation reactions.

In addition to the production of ethanol, bioplastics and other biofuels, the harvested cells can he used for the production of other high value bioproducts, e.g., by further modifying the microbial cellulose-producing cyanobacteria to make other bioproducts, e.g., pharmaceuticals and/or vaccines, vitamins, industrial chemicals, proteins, pigments, fatty acids and their derivatives (such as polyhydroxybutyrate), acylglycerols (as precursors for biodiesel), as well as other secondary metabolites. After each of these steps, the modified-cyanobacteria can then be recycled into the photobioreactors for additional carbon fixation. Furthermore, the products of the processing plant 110 can also be combined with other power sources, e.g., solar, methane, wind, etc., to generate electricity and heat (in addition to recycling any CO₂ released in the processing plant 110), to power the inoculation pool 104 and the photobioreactor 106.

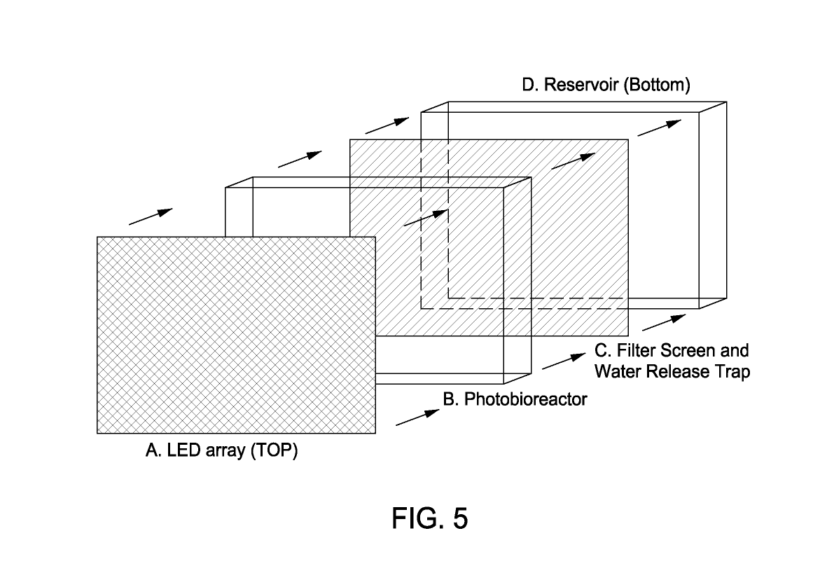

FIG. 5 shows a photobioreactor design for in situ harvest of cyanobacterial saccharides. The photobioreactor complex can be located indoors or underground. A. LED array powered by photovoltaic cells, provides mono or polychromatic light at a pulsed frequencies corresponding to the rate limiting steps of photosynthesis for maximized photosynthetic productivity. Part B is a transparent photobioreactor acting as a growth vessel for cyanobacterial cells. The horizontal orientation of the photobioreactor allows for efficient separation of cells from culture medium by use of gravity and air pressure. Part C is a filter screen combined with a liquid release trap will separate cells from the culture medium. The filter screen will have pore sizes capable of retaining cyanobacterial cells while allowing culture medium to flow into the reservoir. The transfer will be facilitated by gravity and air pressure generated by closing the gas outlet of the photobioreactor. The reservoir, located beneath the photobioreactor, will act to retain culture medium during harvest of saccharides. After harvest, culture medium will be returned to the photobioreactor from the reservoir via pump.

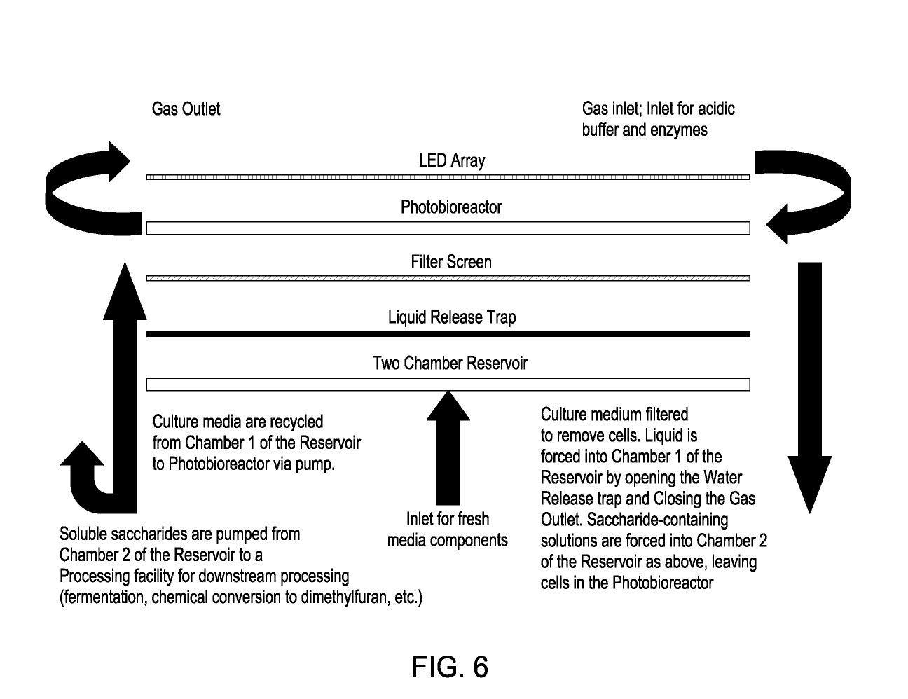

FIG. 6 shows the operation of a photobioreactor complex design for in situ harvest of cyanobacterial saccharides. The LED array, located on top of the photobioreactor complex will supply pulsed mono or polychromatic light for maximum photosynthetic conversion efficiency. Air flow (CO₂, N₂, or ambient air) delivered by the gas inlet during growth periods will serve to deliver carbon and/or nitrogen sources for fixation and created turbulence for maintaining cell suspension. A gas outlet will facilitate the release of waste gasses (O₂ and H₂) that are potentially detrimental to the cyanobacterial growth and relieve excess air pressure from the system during growth phases. Removal of culture media for harvesting of saccharides will be facilitated by the opening of the liquid release trap coupled with closing the gas outlet. The increase in air pressure, combined with gravity, will force the culture medium through the filter which will retain cyanobacterial cells. Cyanobacterial cells can then be resuspended in specific buffer or media designed for cellulose digestion or the direct secretion of saccharides. The saccharide-containing solutions will be drained to chamber 2 of the liquid release trap by the same method described for growth media above. Soluble saccharides will be pumped from chamber 2 of the reservoir to central processing units for downstream conversion processes (e.g., fermentation, chemical conversion to dimethylfuran, etc.). Cells will be resuspended by closing the water release trap and pumping culture medium which has been recombined with fresh media components (e.g., nitrates, phosphates, etc.) from chamber 1 of the reservoir.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

Allen M. (1968). Simple conditions for growth of unicellular blue green algae on plates. J Phycol 4: 1-4.

Golden S S, Brusslan J, Haselkom R. (1988). Mutagenesis of cyanobacteria by classical and gene-transfer-based methods. In Packer L and Glazer A N (eds) Methods in Enzymology ed. Vol. 167 pp 714-727. Academic Press, Inc. New York.

Nobles D R, Romanovicz D K, Brown R M Jr. (2001). Cellulose in cyanobacteria. Origin of vascular plant cellulose synthase? Plant Physiol. 127 (2):529-42.

Roberts E. (1991). Biosynthesis of Cellulose II and Related Carbohydrates PhD thesis. The University of Texas at Austin, Austin.

Roelofsen P A. (1959). The plant cell wall constituents. In: The Plant Cell Wall. Gebrüder Borntraeger (ed). Felengraff and Co. Berlin. pp 1-33.

Sakamoto T and Bryant D A. (1998). Growth at low temperature causes nitrogen limitation in the cyanobacterium Synechococcus sp. PCC 7002. Arch Microbiol. 169: 10-19.

Saxena I M, Kudlicka K, Okuda K, Brown R M Jr. (1994) Characterization of genes in the cellulose synthesizing operon (acs operon) of Acetobacter xylinum: implications for cellulose crystallization. J Bacteriol 176: 5735-5752.

Stevens S E Jr, Patterson C O P, Myers J. (1973). The production of hydrogen peroxide by blue-green algae: a survey. J. Phycol. 9:427-430.

Tel-Or E, Spath S, Packer L, and Mehlhorn R J. (1986). Carbon-13 NMR Studies of Salt Shock-Induced Carbohydrate Turnover in the Marine Cyanobacterium Agmenellum quadruplicatum. Plant Physiol. 82: 646-652.

Updegraff D M. (1969). Semimicro determination of cellulose in biological material. Anal Biochem. 32(3):420-424.

What is claimed is:

1. An isolated halophilic cyanobacterium capable of photosynthetically producing cellulose in brine.

2. The cyanobacterium of claim 1, wherein the cyanobacteria comprises a photosynthetic cyanobacterium capable of growing in brine, and wherein the cyanobacteria produces non-crystalline cellulose and cellulose II as part of its extracellular sheath.

3. The cyanobacterium of claim 2, wherein the extracellular sheath can be digested with cellulose-degrading enzymes.

4. The cyanobacterium of claim 3, wherein the cellulose and its extracellular sheath can be processed into cellulosic ethanol.

5. The cyanobacterium of claim 1, wherein the cyanobacterium can produce cellulose at salt concentrations of greater than 3.5%.

6. The cyanobacterium of claim 1, wherein the cyanobacterium can produce cellulose at salt concentrations of greater than 6%.

7. The cyanobacterium of claim 1, wherein the cyanobacterium is Agmenellum quadruplicatum strain UTEX B2268.

8. The cyanobacterium of claim 3, wherein the cellulose and its extracellular sheath is processed as a renewable feedstock for biofuel production.

9. The cyanobacterium of claim 1, wherein the cyanobacterium can fix CO₂ while producing cellulose and reduce atmospheric CO₂.

10. The cyanobacterium of claim 1, wherein the cyanobacterium can produce cellulose without the use of fresh water.

11. An isolated cyanobacterium capable of producing cellulose in saline environments.

12. The cyanobacterium of claim 11, wherein the cyanobacterium is Agmenellum quadruplicatum UTEX B2268.

13. The cyanobacterium of claim 11, wherein the cyanobacterium produces an extracellular sheath digestible by cellulose-degrading enzymes.

14. The cyanobacterium of claim 11, wherein the cyanobacterium grows at salt concentrations of greater than 4%.

15. A method of producing a photobiomass, comprising:
placing a halophilic cyanobacterium comprising a portion of an exogenous bacterial cellulose operon sufficient to express bacterial cellulose, in brine;
growing the halophilic cyanobacterium under conditions that promote cellulose production; and
separating the cellulose from the brine.

16. The method of claim 15, wherein the separated cellulose and its extracellular sheath are digested with cellulose-degrading enzymes.

17. The method of claim 15, further comprising the step of processing the cellulose into monomers.

18. The method of claim 15, wherein the cellulose and its extracellular sheath are renewable feedstock for biofuel production.

19. The method of claim 15, wherein the cyanobacterium fixes CO₂ and thus atmospheric CO₂.

20. The method of claim 15, wherein the cyanobacterium produces cellulose without the use of fresh water.

21. The method of claim 15, wherein the brine has a salt concentration of greater than 4%.

22. A method of generating carbon credits comprising:
placing a cyanobacterium comprising a portion of an exogenous bacterial cellulose operon sufficient to express bacterial cellulose in CO₂-containing brine; Generating cellulose with the cyanobacterium, wherein CO₂ is fixed into a cellulose biomass; and
calculating the amount CO₂ fixed into the biomass to equate to one or more carbon credit units.

23. The method of claim 22, wherein at least one other carbon is fixed into a cellulose biomass and the at least one other carbon's equate to carbon credit units is included in the calculation.

24. A method for coupled production of cellulose and value added products selected from growing a photosynthetic cyanobacterium capable of growing in brine, and wherein the cyanobacteria produces non-crystalline cellulose and cellulose II as part of its extracellular sheath and expressing one or more genes in the cyanobactierum that produce one or more pharmaceuticals, vaccines, vitamins, industrial chemicals, proteins, pigments, fatty acids and their derivatives (such as polyhydroxybutyrate), acylglycerols (as precursors for biodiesel), as well as other secondary metabolites.
